(19) United States
(12) Patent Application Publication
(10) Pub. No.: US 2015/0301006 A1
(43) Pub. Date: Oct. 22, 2015

(54) Method for approximating the integrated complex error function

(71) Applicants: Brendan Mark Quine, Cookstown (CA); Sanjar Abrarov, Toronto (CA)

(72) Inventors: Brendan Mark Quine, Cookstown (CA); Sanjar Abrarov, Toronto (CA)

(73) Assignee: Thoth Technology Inc., Pembroke (CA)

(21) Appl. No.: 14/258,334

(22) Filed: Apr. 22, 2014

(51) Int. Cl. G01N 33/00

(52) U.S. Cl. CPC G01N 33/0009

(57) ABSTRACT

A method for the high-speed, high-accuracy approximation of spectrally integrated line profiles is described. The approach enables the computation of line-by-line (LBL) radiative transfer at reduced spectral resolution without loss of accuracy. The algorithm provides rapid and accurate computation of area under the line function in a way that preserves spectral radiance and, consequently, radiant intensity. The error analysis we provide shows the high-accuracy of the approximations presented. A comparison of the performance of the method with that of the traditional LBL approaches is given.

### FIELD

The invention relates to a method for the accurate and rapid estimation of the integrated error function (or integrated Voigt function) and other associated functions with application processes including radiative transfer spectroscopy and the computation of spectral radiance.

### BACKGROUND

In high-accuracy radiative transfer models absorption and emission by gases is estimated by summing the accumulated effect of interactions between each electronic transition state of the gas mixture according to quantum mechanical principals and verified by precise laboratory measurements. The ‘line-by-line’ (LBL) method has become established as a powerful tool in the modeling of spectral radiance with applications to disciplines including atmospheric physics and the chemistry of gases [1-11]. Many variations of the LBL computation models such as LBLRTM [2-4], FASCODE [2], A4 [5], GENLN2 [6, 7], LINEPAK [8], GENSPECT [9], have been implemented. Model inter-comparisons and summary descriptions of some common models are given elsewhere [10, 11].

In a LBL model, the accumulated emissivity of a gas mixture is estimated at a specific frequency or wavelength by computing the contribution of all neighbouring electronic transitions or lines. Subject to broadening effects, each transition or line may contribute emissivity at a frequency far from the center frequency of the absorption line. The radiative process can be described through convolution of Lorentz and Doppler broadening functions known as the Voigt function [12-25]. This function has two parameters that capture functional behaviour that are derived from frequency scaled broadening widths associated with the two broadening effects. The parameterization of each line's behaviour is captured in library databases such as HITRAN [26]. The Voigt function cannot be represented in closed form and must be found numerically. Numerical approaches for estimation of the Voigt function have been known for a long time but the development of more efficient approaches remains a hot topic in recent literature [21-25].

The dense line structure of gases, the requirement to compute contributions from each line of a gas mixture at each gas state, and the need for high spectral resolution to capture the absorption process signify that LBL computation is extraordinarily intensive. The recursive computation of the Voigt function typically dominates execution time for LBL codes [9]. The requirement for high spectral resolution is usually driven by a desire to capture sharp variations in emissivity as a consequence of line structure. However, if the spectral resolution of the LBL model is reduced to gain computational acceleration then absorption and emission estimates can underestimate significantly actual values as the sharp line structure in gas emissivity is missed.

Indeed, all LBL codes that use non-integrated line functions are susceptible to this unquantified error at any discrete resolution and empirical data or heuristics must be used to determine the appropriate spectral resolution. Applied to theoretical analyses to determine, for example, the susceptibility of climate to variation in particular atmospheric gases, the computation of gaseous emissivity at specific frequencies, rather than over an integrated spectral interval leads to errors that propagate directly into estimates of radiant energy flux. In all LBL codes where the line function is not spectrally integrated, the energy of the radiative transfer process is not conserved to some extent, breaking one of the most fundamental postulates of physics.

### SUMMARY

The spectrally integrated complex error function provides a practical alternative to the computation of the traditional line function models including the Voigt, Galatry, Rautian-Sobelman and speed-dependent profiles in LBL computations. An algorithm for the computation of the method is presented that provides rapid and accurate results. The approach preserves spectral energy in LBL radiative transfer. It also allows models to be run at reduced spectral resolution without significant loss of accuracy, yielding a considerable computational acceleration in determination of the radiance. A corresponding error analysis reveals the high-accuracy of this approach compared to benchmark regular method.

We show that the spectrally integrated complex error function can find efficient application for LBL computation in a radiative transfer model with reduced spectral resolution or enhanced accuracy. Specifically, the methodology enables the efficient, accurate and rapid computation of the radiance R (v) [Wm⁻² sr⁻¹] (radiant intensity per unit area) following an approach described previously by [27].

We describe a novel computational approximation algorithm based on the Fourier expansion of exponential function. As this technique reduces the requirement for high-spectral resolution, a significant acceleration in the digital estimation of radiance can be achieved.

All example calculations were performed with MATLAB 7.13.0.564 (R2011b) supporting array programming on a typical desktop computer Intel® Quad CPU with RAM 4.00 GB. For computation of the spectral radiance synthetic data, the radiative transfer library GENSPECT is utilized for example calculations [9].

The approximation method is based on implementation of the mean value of the real component of the complex error function or Voigt function K̅(x, y) but can be generalized to the complex component also. The approach enables a more accurate determination of the line broadening profile. The typical implementation procedure requires the determination of the spectral range of computation and the prescribed accuracy that is dependent upon the frequency or wavelength step ΔV=vₗ₊₁−vₗ between two adjacent grid points vₗ and vₗ₊₁. Although the smaller step of the ΔV results in a higher accuracy, it increases drastically the computation time.

In quantitative spectroscopy utilizing the Voigt profile, a wavenumber v is associated to two input parameters x and y while a wavenumber step ΔV is associated to corresponding Δx=xₗ₊₁−xₗ, where xₗ and xₗ₊₁ are two adjacent grid points of the input parameter x. The area occupied under Voigt function within the step Δx, denoted as ΔI, determines the mean value of the Voigt function since K̅(xₗ, yₘ)=ΔI/Δx. In order to implement it, the determination of the function I(x, y) corresponding to the area ΔI is required. This is determined by integrating the Voigt function with respect to the parameter x as

I(x,y)=∫K(x,y)dx.

The I(x, y)-function cannot be found analytically and must be determined numerically by appropriate approximation that provides high accuracy and rapid convergence for fast computation. Once an efficient I(x, y)-function approximation is known, the computation of the mean value of the Voigt function K̅(x, y) is not difficult. In contrast to traditionally applied Voigt function K̅(x, y), the mean value of the Voigt function K̅(x, y) accounts for the area under the curve of the spectral line broadening that is more informative mathematically. As a result, this approach provides higher accuracy at a given spectral resolution. When the results associated to K̅(x, y)-function are found, all other computational procedures in an LBL radiative transfer model remain the same. Due to the interpolation procedure required to connect the grid points, some part of the Voigt function curve is unavoidably missed. As the our approach accounts for the area under the Voigt function, it provides higher accuracy.

The approach is not limited only to Voigt profile, but can be generalized for any kind of the spectral line broadening such as the Rautian-Sobelman, the Galatry or the speed-dependent spectral line broadening profiles. Although we describe the approach as applied to a LBL radiative transfer model, in general, the methodology may be used in other models that utilize spectral-line broadening effects.

The approach described provides a new interpolation methodology for the determination of functional behavior on a discrete grid. In contrast previous methods, the novel interpolation approach applied here is built using the grid-intervals corresponding to the mean values of a function.

### BRIEF DESCRIPTION OF THE DRAWINGS

Table 1. shows example comparison of results of the Brüggemann and Bollig approximation (6) at n=50 and approximation (10) at N=12. The last column shows the relative error values for the Brüggemann and Bollig approximation calculated according to equation (7).

FIG. 1. Shows an example calculation of the Voigt and spectrally integrated Voigt functions at y=0.5. The blue dots are calculated at high spectral resolution Δx=0.0563. The open circles and triangles are calculated at low spectral resolution Δx=3.3 with regular and our methods, respectively.

FIG. 2. shows the synthetic spectral radiance of CO₂ gas for 15-20 km atmospheric layer calculated at high spectral resolution (step is Δv=0.00025 cm⁻¹) with regular method in the spectral range 2000 cm⁻¹ to 2100 cm⁻¹.

FIG. 3. Shows the synthetic spectral radiance of CO₂ gas for 15-20 km atmospheric layer calculated at low spectral resolution at step Δv=0.05 cm⁻¹ with regular method in the spectral range 2000 cm⁻¹ to 2100 cm⁻¹.

FIG. 4. Shows the synthetic spectral radiance of CO₂ gas for 15-20 km atmospheric layer calculated at low spectral resolution at step Δv=0.05 cm⁻¹ with our method in the spectral range 2000 cm⁻¹ to 2100 cm⁻¹.

FIG. 5. Shows contour plots of relative error Δ_{I} in approximation (11) over the domain 0.1≦x≦6 and 0.1≦y≦15 at τₘ=12 and N=40.

FIG. 6. Shows contour plots of relative error Δ_{I} in approximation (13) over the domain 0.1≦x≦15 and 0.1≦y≦15 at τₘ=12 and N=23.

FIG. 7 shows an example of appropriate boundaries for calculation of the spectrally integrated Voigt function.

FIG. 8 shows the contour (a) and 3D (b) plots of relative error Δ_{I} using boundaries for calculation of the spectrally integrated Voigt function.

FIG. 9 shows the contour (a) and 3D (b) plots of logarithm of relative error log₁₀ Δ_{I} using boundaries for calculation of the spectrally integrated Voigt function. The colour bars indicate the orders of the magnitude in relative error.

FIG. 10 shows an example of the determination of the area under the Voigt function at y=0.5: the open circles are the grid points selected for calculation, the open triangles and broken red line are mean values of the Voigt function and corresponding linear interpolation, respectively. The black stepwise curve determines the area under the Voigt function depicted by blue curve.

FIG. 11 shows example synthetic spectral radiance due to CO₂, H₂O, O₂ and CH₄ gases in a wide infrared region.

FIG. 12 shows example synthetic spectral radiance due to CO₂, H₂O, O₂ and CH₄ gases in a narrow infrared region 6470 cm⁻¹ to 6560 cm⁻¹.

FIG. 13 shows the radiance difference AR between our method and an estimate using high-spectral resolution. The resolution in our method is ten times smaller.

FIG. 14 shows an example of the radiance difference ΔR between two regular methods at spectral resolutions at steps Δv_{ref}=0.00025 cm⁻¹ and Δv_{reg}=0.0005.

### DETAILED DESCRIPTION OF THE INVENTION

The spectral radiance [W m⁻² sr⁻¹ (cm⁻¹)⁻¹] originating from the Earth or other celestial bodies can be described accurately by following equation [6, 28].

$${R}_{sp} ( v , {z}_{obs} ) = {R}_{sp} ( v , {z}_{s} ) ⊤ ( v , {z}_{s} , {z}_{obs} ) + {\int }_{{z}_{s}}^{{z}_{obs}} B ( v , T ( z ) ) ⊤ ( v , z , {z}_{obs} ) k ( v , z ) ρ ( z ) d z , \qquad Eqn . ( 1 )$$

where Rₛₚ(v, zₛ) is the spectral radiance from the source point zₛ, T (v, zₛ, z_{obs}) is the optical transmittance from the source zₛ to observation point z_{obs}, T (v, zₛ, z_{obs}) is the optical transmittance from a point z to observation point z_{obs}, B (v,T(z)) is a function of the blackbody radiation and T (z) is the temperature at point z and

$$k ( v ) = {\sum }_{j} {k}_{j} ( v )$$

is the total absorption coefficient due to contribution of each atmospheric gas species j and ρ(z) is the atmospheric gas density.

Optical transmittance is dependent on absorption coefficient since

$$T ( v , {z}_{s} , {z}_{obs} ) = exp ( - {\int }_{{z}_{s}}^{{z}_{obs}} k ( v , z ) ρ ( z ) d z ) .$$

Consequently, the spectral radiance is determined by the line-profile since the absorption coefficient of a gas j is given by [6, 28]

$${k}_{j} ( v ) = {\sum }_{i} {S}_{ij} V ( v , {v}_{i} ) , \qquad Eqn . ( 2 )$$

where Sᵢⱼ is the strength and V(v, v₁) is the line profile with corresponding line centre vᵢ.

For example, the Voigt line profile V(x, y) can be expressed in terms of the Voigt function K(x, y) as follows

$$V ( v , {v}_{i} ) = {K}_{0} ( {α}_{{D}_{i}} ) K ( x ( v , {v}_{i} , {α}_{{D}_{i}} ) , y ( {α}_{{D}_{i}} , {α}_{{L}_{i}} ) ) , \\ where$$

$${K}_{0} ( {α}_{{D}_{i}} ) = \frac{1}{{α}_{{D}_{i}}} \sqrt{\frac{ln 2}{π}}$$

is the normalization parameter, x=\sqrt{ln 2}(v−vᵢ)/α_{D}_{ᵢ}, y=\sqrt{ln 2}α_{L}_{ᵢ}/α_{D}_{ᵢ} and α_{L}_{ᵢ}, α_{D}_{ᵢ} are the Lorentz and the Doppler full widths at half maximum (FWHM) related to line i, respectively.

As we can see from the equations (1) and (2) above, there is a strong relation between the spectral radiance and the line function that describes the spectral broadening in atmospheric emission and absorption. In particular, when the line profile describing the behaviour of the absorption coefficients are known, the spectral radiance can be determined using LBL techniques described elsewhere (see for example Ref. [9]). However, if the spectral resolution is insufficient, the absorption coefficient (2) can be underestimated. This also leads to underestimation of the spectral radiance (1). Since the calculation of the radiance can be made over the wide interval range from vₘᵢₙ to vₘₐₓ, we can expect a significant accumulated error in radiance computation as the area under the spectral radiance curve may consist of large number of the spectral lines i.

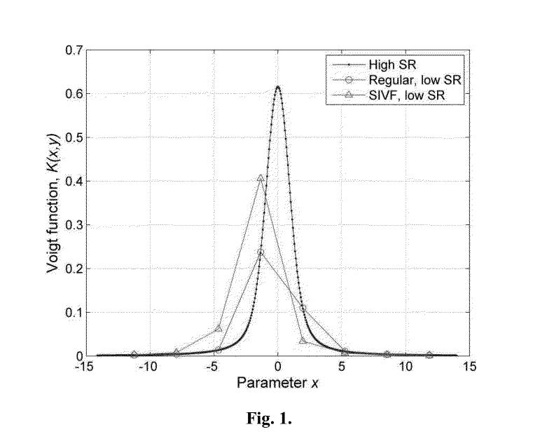

At low spectral resolution the distance between two adjacent points xₗ and xₗ₊₁ is relatively large compared to line structure. When the line centre is located approximately at the middle of two adjacent computation points, the regular method is most inaccurate. FIG. 1 shows the calculated values of the Voigt function where the dots, open circles and open triangles are points obtained with the regular method at high spectral resolution, the regular method at low spectral resolution, and with our method at low spectral resolution, respectively.

At high spectral resolution the interpolated Voigt function accurately approximates the original Voigt function (blue line). At low spectral resolution using the regular method the interpolated Voigt function is quite inaccurate (green line). As we can see in FIG. 1, the interpolated green line misses the significant part of the Voigt function peak. This leads to underestimation of the absorption coefficients and, consequently, to underestimation of radiance.

The problem can be resolved effectively by using the mean value of the line profile, given by

$$\overline{K} ( {x}_{𝓁} , y ) = \frac{1}{Δ x} {\int }_{{x}_{𝓁}}^{{x}_{𝓁 + 1}} K ( x , y ) d x , \qquad Eqn . ( 3 )$$

where Δx=xₗ₊₁−xₗ is the small interval (step) between two adjacent grid points xₗ and xₗ₊₁. As the integration is performed with respect to the parameter x, proportional to the wavenumber (or frequency).

Using this methodology we account for the area under the curve in a way that mathematically is more informative. As a result, application of the approach can resolve effectively problems related to underestimation in absorption, spectral radiance and radiance, particularly where the model resolution is limited due to, for example, the need for computational efficiency.

At low spectral resolution with our method, the interpolated function (red line in FIG. 1) is more accurate as the computed points, shown by open triangles, represent the mean value of the line profile. Significant underestimates of peak radiance can lead to particular problems in regions where the line structure becomes dense. Underestimates of peak line emissivity lead directly to underestimates of absorption due to line partition in LBL calculations. This can be observed by comparing the spectral radiance results computed by regular and our methods using the Voigt line profile as an example.

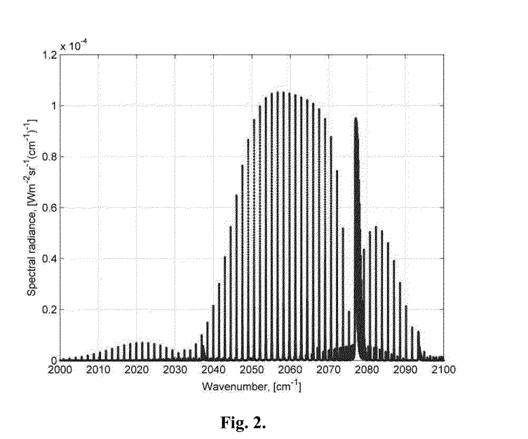

In order to reveal the extent of underestimation, consider a sample computation of the spectral radiance for CO₂ in the stratospheric layer from 15 km to 20 km under typical atmospheric conditions. FIG. 2 shows the spectral radiance for the atmospheric CO₂ gas in the spectral range 2000 cm⁻¹ to 2100 cm⁻¹ by using the regular method at a high resolution step of Δv=0.00025 cm⁻¹, where Δv=vₗ₊₁−vₗ. As we can see the blue dots along the curve mostly overlap each other due to their relatively dense locations representing the computed points.

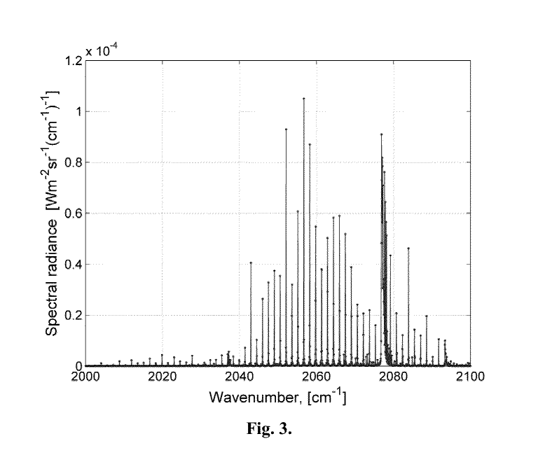

FIG. 3 illustrates the spectral radiance for this atmospheric CO₂ model using the regular method at very low spectral resolution with a step size of only Δv=0.05 cm⁻¹. Comparing FIGS. 2 and 3 we can see that the spectral radiance is strongly underestimated and the shape of the curve is greatly distorted.

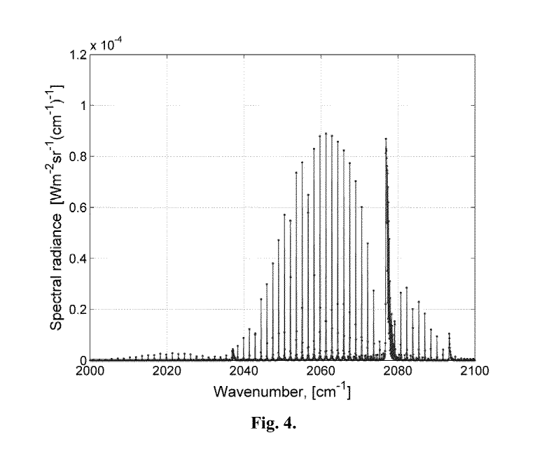

FIG. 4 shows the spectral radiance for the atmospheric CO₂ model using the SIVF method at same step Δv=0.05 cm⁻¹. Comparing FIGS. 2 and 4 one can see that despite the low spectral resolution the underestimation of spectral radiance is much smaller while shape of the curve is well preserved.

Traditionally, an accurate absorption coefficient requires a computation in high spectral resolution. However, this is not desirable as an increase in spectral resolution leads to decrease of the step size Δv and, consequently, to considerably longer computation time. The results obtained in these sample computations indicate that the our method can be significantly advantageous in practical applications. This phenomenon can be explained by the fact that the mean value of the line profile accounts for the area over the step Δx. As a result, the absorption coefficient is computed more accurately since our methodology is based on the mean value of the absorption coefficient within the step Δv according to equations (2) and (3). As a more accurate absorption coefficient results in a more accurate spectral radiance (1), the methodology enables computation of the radiance (or integrated spectral radiance) at reduced spectral resolution.

The approximation for the spectrally integrated Voigt function has been proposed by Brüggemann and Bollig [27]. This approximation is based on Gauss-Hermite quadrature for an integral of kind

$${\int }_{- ∞}^{∞} exp ( - {u}^{2} ) f ( u ) d u \approx {\sum }_{r = 1}^{u} {λ}_{r}^{〈 n 〉} f ( {u}_{r}^{〈 n 〉} ) ,$$

where [symbol] and [symbol] are roots and weights of the Hermite polynomial of n^{th} order Hₙ (u).

The complex probability function is defined by integral [29]

$$W ( z ) = \frac{i}{π} {\int }_{- ∞}^{∞} \frac{exp ( - {t}^{2} )}{z - t} d t ,$$

where z=x+iy is the complex argument. The real part of W (z) is the Voigt function, given by [29]

$$K ( x , y ) = \frac{y}{π} {\int }_{- ∞}^{∞} \frac{exp ( - {t}^{2} )}{{y}^{2} + {( x - t )}^{2}} d t . \qquad Eqn . ( 4 )$$

Consequently, the function

I(x,y)=∫K(x,y)dx=Re[∫W(z)dz′], z′=Re[z]=x,

is the spectrally integrated Voigt function as the integration is taken over the parameter x, associated with wavenumber (or frequency).

Using the Gauss-Hermite quadrature relation above, we can write the following approximation for the complex probability function as [12]

$$W ( z ) \approx \frac{i}{π} {\sum }_{r = 1}^{n} \frac{{λ}_{r}^{〈 n 〉}}{z - {x}_{r}^{〈 n 〉}} . \qquad Eqn . ( 5 )$$

This approximation is accurate when |z| is large. However, it is not useful at |z|<1 since a decrease of |z| drastically deteriorates an accuracy.

Let us take an even order of the Hermite polynomial. Then, applying the symmetric properties of the weights and roots [symbol] = [symbol] [symbol] =− [symbol] , one can rewrite the approximation (5) above as

$$W ( z ) \approx \frac{i}{π} {\sum }_{r = 1}^{n / 2} ( \frac{{λ}_{r}^{〈 n 〉}}{z - {x}_{r}^{〈 n 〉}} + \frac{{λ}_{r}^{〈 n 〉}}{z + {x}_{r}^{〈 n 〉}} ) , \\ n is even .$$

Consequently, after some trivial rearrangements its integration

$$I ( z ) \approx Re [ \frac{i}{π} \int {\sum }_{r = 1}^{n / 2} ( \frac{{λ}_{r}^{〈 n 〉}}{z - {x}_{r}^{〈 n 〉}} + \frac{{λ}_{r}^{〈 n 〉}}{z + {x}_{r}^{〈 n 〉}} ) d {z}^{′} ] , \\ yeilds \\ I ( x , y ) \approx Re [ \frac{i}{π} ln {\prod }_{r = 1}^{n / 2} {\{ {( x + i y )}^{2} + {( {x}_{r}^{〈 n 〉} )}^{2} \}}^{{λ}_{r}^{〈 n 〉}} ] + const . , \\ n is even . \qquad Eqn . ( 6 )$$

This is a simplified representation of the Brüggemann and Bollig approximation [27]. The magnitude of the constant on the right side of the approximation (6) can be readily found by comparing the result of computation with a reference value at arbitrary |z|>>1. Using this technique we found that the constant is 0.886226925452758 (note that 0.886226925452758\sqrt{π}/2). The Brüggemann and Bollig approximation (6) is highly accurate when |z| is large.

However, as it is derived from approximation (5) above, its accuracy also drastically deteriorates as |z| decreases. This can be seen from Table 1 showing results of the approximation (6) together with highly accurate reference values. The last column in Table 1 illustrates the relative error that is defined according to equation

$${Δ}_{I} = \frac{\vert I ( x , y ) - {I}_{ref .} ( x , y ) \vert}{{I}_{ref .} ( x , y )} , \qquad Eqn . ( 7 )$$

where I_{ref} (x, y) is the reference (reference values are generated using approximation (10) that will be described later).

As one can see from this table, despite of very high order of the Hermite polynomial (it is taken to be n=50) the accuracy of the approximation (6) deteriorates and ultimately fails in computation with decreasing |z| (see the last three rows in Table 1). The Brüggemann and Bollig approximation (6) cannot cover the important range of the input parameters x and y. This motivated us to look for another approach in I(x, y)-function approximation.

The Voigt function in form of (4) is most common in literature. However, in our work we also apply an alternative representation of the Voigt function that can be obtained by Fourier transform [29]

$$K ( x , y ) = \frac{1}{\sqrt{π}} {\int }_{0}^{∞} exp ( - {t}^{2} / 4 ) exp ( - yt ) cos ( xt ) d t , \\ y > 0. \qquad Eqn . ( 8 )$$

Consequently, the integration of the Voigt function over the parameter x is represented as follows

$$\begin{matrix}I ( x , y ) = \int K ( x , y ) d x \\ = \frac{1}{\sqrt{π}} {\int }_{0}^{∞} exp ( - {t}^{2} / 4 ) exp ( - yt ) \frac{sin ( xt )}{t} d t \\ = \frac{x}{\sqrt{π}} {\int }_{0}^{∞} exp ( - {t}^{2} / 4 ) exp ( - yt ) sin c ( xt ) d t .\end{matrix} \qquad Eqn . ( 9 )$$

While the parameter y=0 the integral (9) is not problematic since substitution of equation (4) into the limit

$$I ( x , y = 0 ) = {lim}_{y \to 0} \int K ( x , y ) d x$$

leads immediately to

$$I ( x , y = 0 ) = \int exp ( - {x}^{2} ) d x = \frac{\sqrt{π}}{2} erf ( x )$$

and the calculation of the error function of a real argument x can be performed very rapidly by a built-in function available in most programming languages.

However, the problem appears at nonzero y as the integral (9) has no analytical solution in closed form and, therefore, must be solved numerically [27].

One of the approximations is given by

$$I ( x , y ) \approx \frac{x}{{2}^{N - 1}} {\sum }_{n = 1}^{{2}^{N - 1}} k ( \frac{2 n - 1}{{2}^{N}} x , y ) . \qquad Eqn . ( 10 )$$

Despite the simplicity and high-accuracy, the application of approximation (10) is not optimal for practical use. In particular, in order to obtain a reasonable accuracy exceeding ˜10⁻⁶ within domain −6≦x≦6, the integer number N should be equal or greater than 7. This requires that the number of the Voigt functions in approximation (10) should be at least equal to 2⁷⁻¹=64. As a result, the computation becomes approximately 2^{N-1} times longer compared to the computation of the Voigt function alone.

This problem can be effectively resolved by Fourier expansion, similar to that proposed in our previous works [20, 22]. Specifically, we can apply an approximation given by

$$I ( x , y ) \approx \frac{\sqrt{π}}{2} ( {b}_{0} ( x ) \frac{1 - {e}^{- {τ}_{m} y}}{2 {τ}_{m} y} - {τ}_{m} y {{\sum }_{n = 1}}^{N} {b}_{n} ( x ) \frac{{( - 1 )}^{n} {e}^{- {τ}_{m} y} - 1}{{n}^{2} {π}^{2} + {τ}_{m}^{2} {y}^{2}} ) , \qquad Eqn . ( 11 )$$

where the Fourier expansion coefficients are

$${b}_{n} ( x ) = erf ( \frac{n π}{{τ}_{m}} + x ) - erf ( \frac{n π}{{τ}_{m}} - x ) . \qquad Eqn . ( 12 )$$

The approximation series (11) is considerably faster in computation than the previous approach. It converges perfectly well at |x|̂1, but larger values of the parameter x require larger integer N. For instance, in order to sustain the high-accuracy at |x|≦6 the integer N should be increased up to 33.

Another way to approximate equation (9) is to use a remarkable property of the sinc function [30]. Particularly, an argument of the sinc function can be reduced by factor of 2^{M}. For example, taking M equal to 3 reduces argument of the sinc function by factor of 8. As a result, this technique enables us to keep the integer N=23 that is more than sufficient to cover the region |x|≦6. The corresponding approximation based on reduced argument of the sinc function by factor of 8 is given by

$$I ( x , y ) \approx \frac{1}{4} Re [ {I}_{1} ( \frac{x}{8} , y ) + {I}_{3} ( \frac{3 x}{8} , y ) + {I}_{5} ( \frac{5 x}{8} , y ) + {I}_{7} ( \frac{7 x}{8} , y ) ] , \qquad Eqn . ( 13 )$$

where the terms, I₁(x, y)−I₇(x, y) are defined according to

$${I}_{p} ( x , y ) = 2 i \sqrt{π} ( {b}_{0} ( x / p ) \frac{1 - {e}^{i {τ}_{m} ( x + i y )}}{{τ}_{m} ( x + i y )} + 2 {τ}_{m} ( x + i y ) {\sum }_{n = 1}^{N} {b}_{n} ( x / p ) \frac{{( - 1 )}^{n} {e}^{i {τ}_{m} ( x + i y )} - 1}{{n}^{2} {π}^{2} - {{τ}_{m}^{2} ( x + i y )}^{2}} ) , \qquad Eqn . ( 14 )$$

where pε{1,3,5,7}. An example and some details related to the terms, I₁(x, y)-I₇(x, y) is presented later.

While y≧1 or |x|≧4.5 the following approximation can be effectively used

$$I ( x , y ) \approx \frac{1}{{τ}_{m}} arctan ( \frac{x}{y} ) + \frac{1}{2 \sqrt{π}} {\sum }_{n = 1}^{N} {a}_{n} [ arctan ( \frac{x + n π / {τ}_{m}}{y} ) + arctan ( \frac{x - n π / {τ}_{m}}{y} ) ] , \qquad Eqn . ( 15 )$$

where the Fourier expansion coefficients are given by

$${a}_{n} \approx \frac{2 \sqrt{π}}{{τ}_{m}} exp ( - \frac{{n}^{2} {π}^{2}}{{τ}_{m}^{2}} ) . \qquad Eqn . ( 16 )$$

As the approximation (15) utilizes the arctangent function, it is essentially more rapid than approximations (11) or (13). The derivation of this approximation is given later.

Lastly, for |x+iy/1.54545|>11 the following simple approximation can be applied

$$I ( x , y ) \approx \frac{arctan ( x / y )}{\sqrt{π}} - \frac{xy}{2 \sqrt{π} {( {x}^{2} + {y}^{2} )}^{2}} . \qquad Eqn . ( 17 )$$

Despite the presence of the arctangent function, the computational test shows that this approximation is as rapid as a rational function of the power 4. The derivation of approximation (17) is given later.

In order to perform error analysis we can apply equation (7). The reference I_{ref} (x, y) for relative error for this equation can be obtained, for example, according to approximation (10) as superposition of the Voigt functions K((2n−1)x/2^{N}, y) at N≧12 with help of well-known and highly accurate Algorithm 680 [15, 16] (or Algorithm 916 [21]). Alternatively, the functions K ((2n−1)x/2^{N}, y) can also be obtained by taking the real part of the complex error function as proposed in our recent works [22, 23]

$$Eqn . ( 18 )$$

$$\begin{matrix}w ( z ) \approx \frac{i}{2 \sqrt{π}} [ {\sum }_{n = 0}^{N} {a}_{n} {τ}_{m} ( \frac{1 - {e}^{i ( n π + {τ}_{m} z )}}{n π + {τ}_{m} z} - \frac{1 - {e}^{i ( - n π + {τ}_{m} z )}}{n π - {τ}_{m} z} ) - {a}_{0} \frac{1 - {e}^{i {τ}_{m} z}}{z} ] \\ = i \frac{1 - {e}^{i {τ}_{m} z}}{{τ}_{m} z} + i \frac{{τ}_{m}^{2} z}{\sqrt{π}} {\sum }_{n = 1}^{N} {a}_{n} \frac{{( - 1 )}^{n} {e}^{i {τ}_{m} z} - 1}{{n}^{2} {π}^{2} - {τ}_{m}^{2} {z}^{2}} ,\end{matrix}$$

where aₙ are the Fourier expansion coefficients that can be found according to equation (16) at τₘ=12 and N=23.

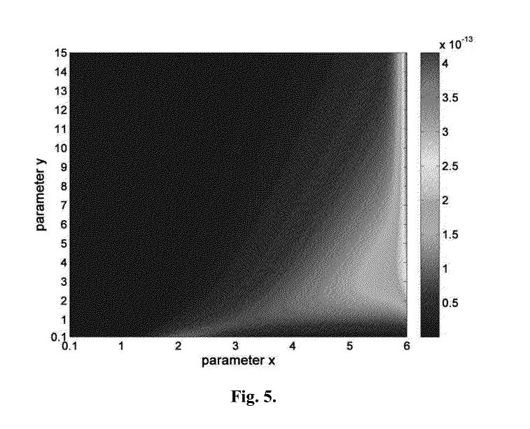

The Algorithm 680 and proposed approximation (18) of the complex error function generate values of the Voigt function matching up to the last decimal digits for the range of input parameters 0≦x≦40000 and 10⁻⁶≦y≦10⁴ required typically for atmospheric spectroscopy [9, 18]. Therefore FIGS. 5, 6, 8 and 9 showing our results of the relative error are visually identical irrespective of whether we use the reference I_{ref} (x, y) based on Algorithm 680 or approximation (18). However, to the best of our knowledge, the algorithm built on approximation (18) is the most rapid for computation compared with those providing an accuracy better than 10⁻⁹ in the Humlí\check{c}ek regions 3 and 4 (see Refs. [13, 17, 18] for details of regions in complex plane).

FIG. 5 shows the relative error Δ_{I} for approximation (11) at τₘ=12 and N=40. As we can see, the relative error within the range |x|≦6 is 10⁻¹² indicating a high-accuracy. Beyond this range the accuracy drastically decreases and the only way to sustain it is to increase further the integer N. However, this is not desirable as the increase of N drastically increases computation time.

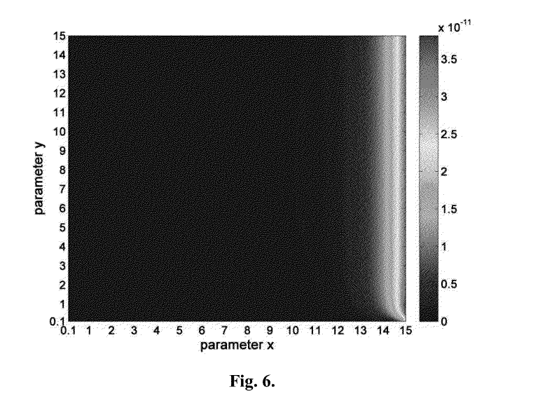

FIG. 6 shows the relative error for approximation (13) at |x|≦15 and N=23. The corresponding accuracy is 10⁻¹⁰. As we can see, the range for x is essentially wider. However, in the range |x|≦6 approximation (13) is slower than (11) by a factor about 4.

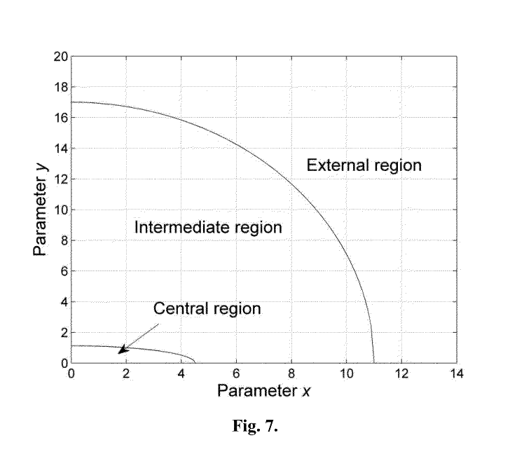

FIG. 7 shows example regions where the corresponding I(x,y)-function approximations above are used for the computation. There are two ellipses in the 1^{st} quadrant. The semi-major and semi-minor axes of the small ellipse along x and y axes are equal to 4.5 and 1.125, respectively. The large ellipse has the values of the semi-major and semi-minor along x and y axes that are equal to 17 and 11, respectively. These ellipses separate the complex plane to the central, intermediate and external regions.

The central region is located inside the small ellipse and bounded by |x+iy/0.25|≦4.5. The intermediate region is located inside the large ellipse without intercepting the small ellipse and bounded by |x+iy/1.54545|≦11 and |x+iy/0.25|>4.5. Lastly, the external region is located outside the large ellipse and bounded by |x+iy/1.54545>>11.

The input parameters falling into the intermediate region use approximation (15) for computation. As an alternative, the Brüggemann and Bollig approximation (6) can also be applied for intermediate region. However, approximation (15) is preferable as it has enhanced accuracy and is essentially faster in computation (the Brüggemann and Bollig approximation (6) requires a repetitive exponentiation procedure with non-integer floating point values [symbol] that decelerates calculation). The input parameters falling into central region can be used for computation either by approximation (11) or (13). Although the approximation (13) is slower than (11), the central region area is considerably smaller than the area occupied by intermediate region. As a result, the fraction of input parameters falling into the central region is small. Consequently, application of the approximation (13) does not decelerate the computation significantly. The input parameters falling into external region are used for computation with approximation (17) that is very rapid in computation. Once I(x, y) values are found, the mean values of the Voigt function can be calculated according to equation (3).

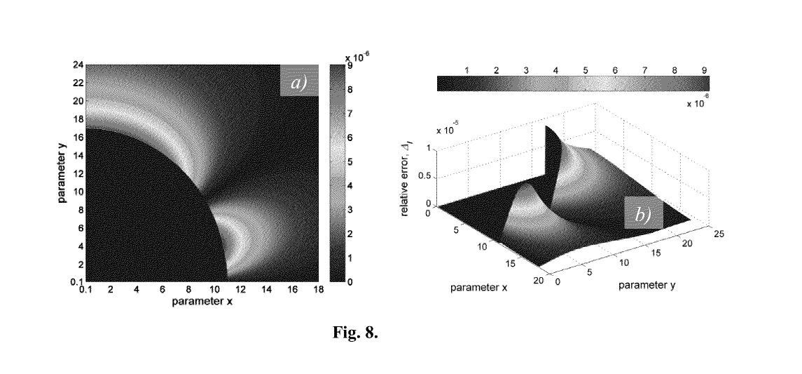

FIGS. 8a and 8b illustrate contour and 3D plots of the relative error Δ_{I} for the computational scheme described above (see also FIG. 7). In the external region Δ_{I} is better than 10⁻⁵. Although Δ_{I} is not highly accurate, this is not critical for the parameters falling into external region at low or moderate spectral resolution. Furthermore, the Voigt function is very well-behaved at the larger values of the input parameters x and y. Consequently, when the condition |x+iy/1.545451>11 for the input parameters is satisfied, then at Δx≦0.5 the mean value of the Voigt function can be found very precisely as average within the step Δx as:

K̅(xₗ,yₘ)≈K(xₗ+Δx/2,yₘ). Eqn. (19)

Thus, if the step Δx is small enough we can simply avoid the use of approximation (17) for the external region since the mean value of the Voigt function can be determined directly from the Voigt function according to equation (19). It should be noted, however, that the equation (19) cannot be used for the input parameters falling into the central and/or intermediate regions as the accuracy drastically deteriorates at smaller values of the input parameters x and y. Such behaviour in some sense is similar to that of the Voigt function; while in the Humlí\check{c}ek regions 1 and 2 the simple rational function approximations can be used, the Humlí\check{c}ek regions 3 and 4 require the higher order polynomial in the rational functions for reasonably accurate computation [17, 18].

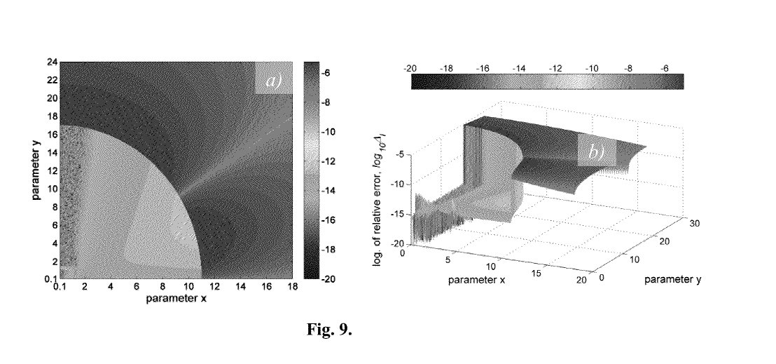

In the central region, the generated results are highly accurate. In order to emphasize the features inside the central and intermediate regions we apply a logarithmic relative error scale. FIGS. 9a and 9b show contour and 3D plots for log₁₀ Δ_{I}. Therefore in this figures the color bars depict the order of the accuracy. The central region was computed by approximation (11) at τₘ=12 and N=33.

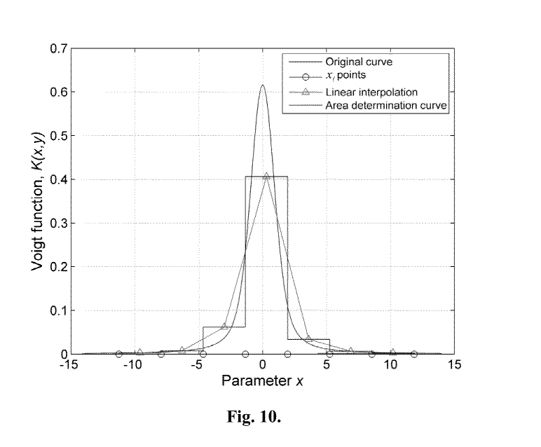

FIG. 10 illustrates our area-integrated approach. Open circles show the grid points xₗ chosen for calculation of the Voigt function. The mean value points, shown by open red triangles, are relocated at the middle of the intervals xₗ as K̅(xₗ,yₘ)→ K̅(xₗ+Δx/2, yₘ) while the red line depicts a linear interpolation connecting these points. The area under the Voigt function for the same spectral intervals is shown by a stepwise black line. Since the pre-computed points K̅(xₗ+Δx/2, yₘ) represent the mean value of the Voigt function within each interval Δx, the area under the stepwise function (broken black line) is always equal to the area of the Voigt function (blue line). The computation of the area under stepwise function is straightforward and rapid.

Once the spectrally integrated function is known the determination of the mean value of the function is not difficult. Since I(x, y) integrates over the spectral domain (in wavenumbers), the area under the line function curve between two arbitrary points xₗ and xₗ₊₁ is just a difference I(xₗ₊₁, yₘ)−I(xₗ, yₘ), where yₘ is some fixed parameter. Therefore to determine the mean value of the line function we can write

$$\overline{K} ( {x}_{𝓁} , {y}_{m} ) = \frac{I ( {x}_{𝓁 + 1} , {y}_{m} ) - I ( {x}_{𝓁} , {y}_{m} )}{Δ x} = \frac{Δ I}{Δ x} .$$

In array programming this procedure does not require double computation of the I(x, y) function at x=xₗ and x=xₗ₊₁. If an array I contains the numbers corresponding to the I(x, y) function, then the array K̅ for the mean values of the line function can be assigned as

$$\overline{K} := \frac{I ( 2 \text{:} end ) - I ( 1 \text{:} end - 1 )}{x ( 2 \text{:} end ) - x ( 1 \text{:} end - 1 )} ,$$

where x is the array with numbers corresponding to the variable x. As we can see from this approach, a single array I is sufficient to determine the array K̅.

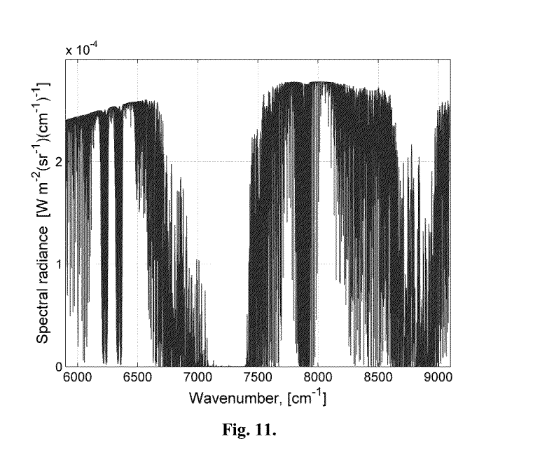

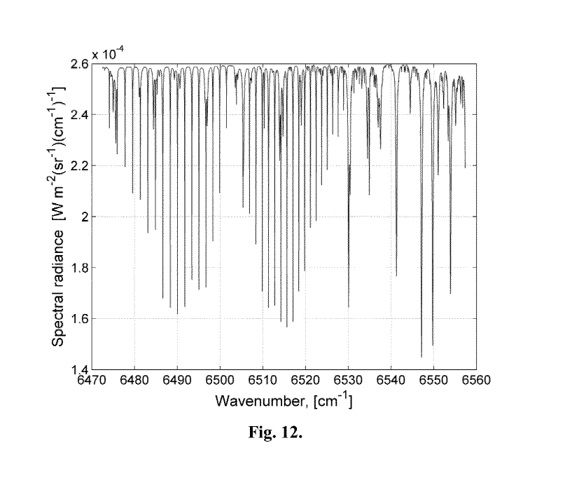

FIG. 11 shows a synthetic spectral radiance simulated for nadir view observation from the top of the atmosphere for a wide near infrared region. In FIG. 12 we select as an example a narrow region, say from 6470 cm⁻¹ to 6560 cm⁻¹, were strong CO₂ absorption lines are clearly observed.

The computational advantage of the method is obtained due to the reduction in the spectral resolution required for the computation of high-accuracy results. In order to illustrate this advantage we compare results generated by the method at a lower spectral resolution with those computed using a regular non-integrated line model at higher spectral resolution.

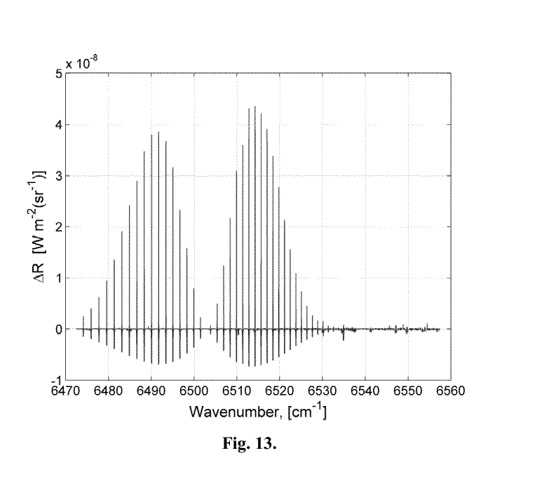

FIG. 13 shows the difference between the values of the radiance

ΔR=R_{SIVF}(v)−R_{ref.}(v)

where R_{SIVF} (v) is the radiance computed by the method at step Δv_{SIFV}=0.0025 cm⁻¹ and R_{ref.}(v) is the high resolution radiance computed by regular method at step Δv_{ref.}=0.00025 cm⁻¹ using the SIVF as an example.

As Δv_{ref.}<<Δv_{SIVF} the radiance R_{ref.}(v) is interpolated through multiple radiance points above Δv_{SIVF} and then computed as the mean value for this step Δv_{SIVF}. As we can see despite the decrease in resolution by factor Δv_{SIVF}/Δv_{ref.}=10, the difference in ΔRₘₐₓ does not exceed 5×10⁻⁸ W m⁻² sr⁻¹.

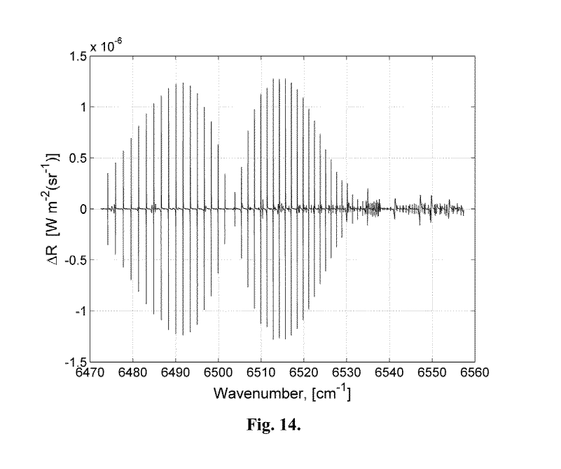

FIG. 14 shows the difference between the values of the radiance

ΔR=R_{reg.}(v)−R_{ref.}(v)

where R_{ref.}(v) is radiance computed by regular method at step Δv_{reg.}=0.0005 cm⁻¹.

Similar to the procedure above, the radiance R_{ref.}(v) is interpolated through radiance points above Δv_{reg.} and then computed as the mean value for this step Δv_{reg.}. Although the decrease in resolution Δv_{SIVF}/Δv_{ref.}=2 is not significant, the difference ΔRₘₐₓ is about 1.5×10⁻⁶ W m⁻² sr⁻¹. Comparing ΔRₘₐₓ with previous value, one can see that despite the considerably lower resolution used by our method it, nevertheless, provides an accuracy that is better by two orders of magnitude.

The increase of the computation time with increasing the resolution is nonlinear. Taking this into account and considering the fact that for the same size of input array our method is slower in computation than the regular method by factor about 1.5, we gain an acceleration in computation of radiance by a factor exceeding 5. This is possible to achieve since the method needs a significantly lower spectral resolution. Specifically, in this example we used the SIVF with spectral resolution that is 10 times lower to obtain practically same accuracy in computation of the radiance. The derivation steps required for our new approach are now presented below.

Using Viète's infinite product for sinc function [30]

$$sin c ( xt ) ≡ {\prod }_{n = 1}^{∞} cos ( \frac{xt}{{2}^{n}} ) \qquad Eqn . ( 20 )$$

and cosine product-to-sum identity

$${\prod }_{n = 1}^{N} cos ( \frac{xt}{{2}^{n}} ) ≡ \frac{1}{{2}^{N - 1}} {\sum }_{n = 1}^{{2}^{N - 1}} cos ( \frac{2 n - 1}{{2}^{N}} xt ) \qquad Eqn . ( 21 )$$

we can rewrite equation (9) as

$$I ( x , y ) = {lim}_{N \to ∞} \frac{x}{{2}^{N - 1} \sqrt{π}} {\sum }_{n = 1}^{{2}^{N - 1}} {\int }_{0}^{∞} exp ( - {t}^{2} \text{/} 4 ) exp ( - yt ) cos ( \frac{2 n - 1}{{2}^{N}} xt ) d t . \qquad Eqn . ( 22 )$$

Comparing equations (8) with (22) one can see that I(x,y) function can be represented as a superposition of the Voigt functions. Thus, truncating the series (22) over the integer N we can approximate I(x, y) function in form of equation (10).

The expansion of multiplication of the exponential and sinc functions exp (−t²/4) sinc (xt) into Fourier series is given by

$$exp ( - {t}^{2} \text{/} 4 ) sin c ( xt ) = {\sum }_{n = 0}^{∞} {a}_{n} cos ( \frac{n π}{{τ}_{m}} t ) - \frac{{a}_{0}}{2} , - {τ}_{m} \leq t \leq {τ}_{m} , \qquad Eqn . ( 23 )$$

where τₘ is the margin value yet to be determined and

$${a}_{n} = \frac{1}{{τ}_{m}} {\int }_{- {τ}_{m}}^{{τ}_{m}} exp ( - \frac{{t}^{2}}{4} ) sin c ( xt ) cos ( \frac{n π}{{τ}_{m}} t ) d t , \qquad Eqn . ( 24 )$$

are the expansion coefficients.

Since the sinc function decays slower with decreasing x, we should consider exp (−t²/4) sinc (xt)|_{x→0}→exp (−t²/4) in order to determine the proper margin value τₘ. As it has been shown previously for the exponential function exp (−t²/4) expanded into Fourier series, the margin value can be taken τₘ≧12 [20, 22, 31].

The definite integral in equation (24) has no analytical solution. However, this problem can be effectively overcome by replacing the upper and lower integral boundaries −τₘ and τₘ by −∞ and ∞, respectively. Such replacement is absolutely justified because the multiplication exp (−t²/4) sinc (xt) damps very rapidly to zero with increasing t even when x→0 (see also [20, 22] for comparison).

This leads to the following approximation

$${a}_{n} = \frac{1}{{τ}_{m}} {\int }_{- ∞}^{∞} exp ( - \frac{{t}^{2}}{4} ) sin c ( xt ) cos ( \frac{n π}{{τ}_{m}} t ) d t . \qquad Eqn . ( 25 )$$

The integral in approximation equation (25) has an analytical solution. As a consequence, the expansion coefficients can be computed as

$${a}_{n} ( x ) \approx \frac{π}{2 {τ}_{m} x} [ erf ( \frac{n π}{{τ}_{m}} + x ) - erf ( \frac{n π}{{τ}_{m}} - x ) ] . \qquad Eqn . ( 26 )$$

Substituting equations (23) into (9) and replacing the upper limit by a margin value τₘ and integrating with respect to x yields the following approximation

$$I ( x , y ) \approx \frac{x}{\sqrt{π}} ( {\sum }_{n = 0}^{N} {\int }_{0}^{{τ}_{m}} {a}_{n} ( x ) exp ( - yt ) cos ( \frac{n π}{{τ}_{m}} t ) d t - {\int }_{0}^{{τ}_{m}} \frac{{a}_{0} ( x )}{2} exp ( - yt ) d t ) . \qquad Eqn . ( 27 )$$

Each term in the series equation (27) is now integrable. Consequently, the integral of the Voigt function can be approximated as

$$I ( x , y ) \approx \frac{x}{\sqrt{π}} ( {\sum }_{n = 0}^{N} {a}_{n} ( x ) \frac{{τ}_{m}^{2} y - {e}^{- {τ}_{m} y} ( {τ}_{m}^{2} y cos ( n π ) + n {πτ}_{m} sin ( n π ) )}{{n}^{2} {π}^{2} + {τ}_{m}^{2} {y}^{2}} - {a}_{0} ( x ) \frac{1 - {e}^{- {τ}_{m} y}}{2 y} ) . \qquad Eqn . ( 28 )$$

Taking into account that cos (nπ)=(−1)ⁿ and sin (nπ)=0, the equation (25) can be further simplified as

$$I ( x , y ) \approx \frac{x}{\sqrt{π}} ( {a}_{0} ( x ) \frac{1 - {e}^{- {τ}_{m} y}}{2 y} - {τ}_{m}^{2} y {\sum }_{n = 1}^{N} {a}_{n} ( x ) \frac{{( - 1 )}^{n} {e}^{- {τ}_{m} y} - 1}{{n}^{2} {π}^{2} + {τ}_{m}^{2} {y}^{2}} ) . \qquad Eqn . ( 29 )$$

At x=0 the use of equation (26) becomes inappropriate due to uncertainty zero over zero. To resolve this, we redefine the expansion coefficients by set (12). This correspondingly modifies the approximation equation (29) as shown in equation (11).

From the Viète's infinite product (20) we can obtain the following identity

$$sin c ( xt ) ≡ sin c ( \frac{xt}{{2}^{M}} ) {\prod }_{m = 1}^{M} cos ( \frac{xt}{{2}^{m}} ) , M ∈ \{ 1 , 2 , 3 … \} . \qquad Eqn . ( 30 )$$

As we can see, the argument of the sinc function in equation (30) decreases with increasing integer M and we can apply this remarkable property of the sinc function to reduce the integer N that determines the number of the summation terms. Substituting identity (30) into equation (9) results in

$$I ( x , y ) = \frac{x}{\sqrt{π}} {\int }_{0}^{∞} exp ( - {t}^{2} \text{/} 4 ) exp ( - yt ) sin c ( \frac{x}{{2}^{M}} t ) {\prod }_{m = 1}^{M} cos ( \frac{x}{{2}^{m}} t ) d t . \qquad Eqn . ( 31 )$$

The integer M in should not be large to avoid excessive quantity of the cosine multipliers in approximation. For instance, the values for M equal to 3 and 4 are found very useful for the practical applications.

Let us consider M=3. Using identity (30) and product-to-sum formula (21), we can write

$$\begin{matrix}sin c ( xt ) = sin c ( \frac{x}{{2}^{3}} t ) {\prod }_{m = 1}^{3} cos ( \frac{x}{{2}^{m}} t ) \\ = sin c ( \frac{x}{8} t ) \frac{1}{4} [ cos ( \frac{x}{8} t ) + cos ( \frac{3 x}{8} t ) + cos ( \frac{5 x}{8} t ) + cos ( \frac{7 x}{8} t ) ] .\end{matrix} \qquad Eqn . ( 32 )$$

Expanding

$$exp ( - {t}^{2} \text{/} 4 ) sin c ( \frac{x}{8} t )$$

into Fourier series in a similar way discussed above, replacing the upper limit by the margin value τₘ and substituting the identity (32) into (31), we obtain

$$I ( x , t ) \approx \frac{x}{\sqrt{π}} \{ {\sum }_{n = 0}^{N} {\int }_{0}^{{τ}_{m}} {a}_{n} ( x \text{/} 8 ) {e}^{- yt} \frac{1}{4} [ … ] cos ( \frac{n π}{{τ}_{m}} t ) d t - {\int }_{0}^{{τ}_{m}} \frac{{a}_{0} ( x \text{/} 8 )}{2} {e}^{- yt} \frac{1}{4} [ … ] d t \} , \qquad Eqn . ( 33 )$$

where [ . . . ] denotes a shorthand notation for

$$[ cos ( \frac{x}{8} t ) + cos ( \frac{3 x}{8} t ) + cos ( \frac{5 x}{8} t ) + cos ( \frac{7 x}{8} t ) ]$$

and in accordance with (26) the expansion coefficients are now

$${a}_{n} ( x \text{/} 8 ) \approx \frac{π}{2 {τ}_{m} x} [ erf ( \frac{n π}{{τ}_{m}} + \frac{x}{8} ) - erf ( \frac{n π}{{τ}_{m}} - \frac{x}{8} ) ] . \qquad Eqn . ( 34 )$$

Each integral term in equation (33) is readily integrable. However, due to uncertainty zero over zero at x=0 in approximation (34) the same problem discussed earlier appears again in computation. To resolve it we can apply the expansion coefficients bₙ (x/8) instead of aₙ (x/8). This slightly modifies the series (33) as

$$I ( x , y ) \approx \frac{\sqrt{π}}{2 {τ}_{m}} \{ {\sum }_{n = 0}^{N} {\int }_{0}^{{τ}_{m}} {b}_{n} ( x \text{/} 8 ) {e}^{- yt} \frac{1}{4} [ … ] cos ( \frac{n π}{{τ}_{m}} t ) d t - {\int }_{0}^{{τ}_{m}} \frac{{b}_{0} ( x \text{/} 8 )}{2} {e}^{- yt} \frac{1}{4} [ … ] d t \} ,$$

ultimately leading to approximation (13). In approximation (13) the value of the integer N=23 is sufficient and provides the highly accurate results in the central region shown in FIG. 7.

As an example, consider the third term on the right hand side of approximation (13). Since the corresponding integer p is 5, the replacement in argument x→5x/8 in equation (12) yields (see also equation (13))

$${b}_{n} ( x \text{/} 5 ) \to {b}_{n} ( \frac{5 x \text{/} 8}{5} ) = {b}_{n} ( x \text{/} 8 )$$

$$and$$

$${I}_{5} ( \frac{5 x}{8} , y ) = 2 i \sqrt{π} ( {b}_{0} ( x \text{/} 8 ) \frac{1 - {e}^{i {τ}_{m} ( 5 x \text{/} 8 + iy )}}{{τ}_{m} ( 5 x \text{/} 8 + iy )} + 2 {τ}_{m} ( \frac{5 x}{8} + iy ) {\sum }_{n = 1}^{N} {b}_{n} ( x \text{/} 8 ) \frac{{( - 1 )}^{n} {e}^{i {τ}_{m} ( 5 x \text{/} 8 + iy )} - 1}{{n}^{2} {π}^{2} - {{τ}_{m}^{2} ( 5 x \text{/} 8 + iy )}^{2}} )$$

where in accordance to equation (12) the expansion coefficients are

$${b}_{n} ( x \text{/} 8 ) = erf ( \frac{n π}{{τ}_{m}} + \frac{x}{8} ) - erf ( \frac{n π}{{τ}_{m}} - \frac{x}{8} ) . \qquad Eqn . ( 35 )$$

All other terms I₁(x/8, y), I₃(3x/8, y) and I₇(7x/8, y) can be found similarly.

It should be noted that the expansion coefficients remain same in all these terms due to relation

$${b}_{n} ( \frac{px \text{/} 8}{p} ) = {b}_{n} ( x \text{/} 8 ) .$$

Since the computation of the expansion coefficients is the most time consuming, a single use of equation (35) significantly saves time—the array bₙ (x/8) being computed only once can be further substituted into all summation terms on the right hand side of approximation (13).

The exponential function can be approximated by Fourier expansion [20, 22] as follows

$$exp ( - \frac{{t}^{2}}{4} ) \approx - \frac{{a}_{0}}{2} + {\sum }_{n = 0}^{N} {a}_{n} cos ( \frac{n π}{{τ}_{m}} t ) , - {τ}_{m} \leq t \leq {τ}_{m} , \qquad Eqn . ( 36 )$$

where the Fourier expansion coefficients are given by equation (16).

The expansion (36) can also be obtained by Poisson summation formula in a way described in publication [32]. This Fourier expansion is valid only within the domain −τₘ≦t≦τₘ. As the exponential function exp (−t²/4) is rapidly decreasing function, we can write

$$I ( x , y ) \approx - \frac{{a}_{0} x}{2 \sqrt{π}} {\int }_{0}^{{τ}_{m}} exp ( - yt ) sin c ( xt ) d t + \frac{x}{\sqrt{π}} {\sum }_{n = 0}^{N} {\int }_{0}^{{τ}_{m}} {a}_{n} cos ( \frac{n π}{{τ}_{m}} t ) exp ( - yt ) sin c ( xt ) d t . \qquad Eqn . ( 37 )$$

This approximation contains two damping functions exp (−yt) and sine (xt) that effectively suppresses the integrand in each summation term when the parameters y and x are sufficiently large. Therefore we can assume that if y and x are large enough, the upper limit of integral can be extended to infinity without any loss of accuracy, i.e.:

$${\int }_{0}^{{τ}_{m}} cos ( \frac{n π}{{τ}_{m}} t ) exp ( - yt ) sin c ( xt ) d t \approx {\int }_{0}^{∞} cos ( \frac{n π}{{τ}_{m}} t ) exp ( - yt ) sin c ( xt ) d t .$$

As a result, the approximation (37) above can be rewritten as

$$I ( x , y ) \approx - \frac{{a}_{o} x}{2 \sqrt{π}} {\int }_{0}^{∞} exp ( - yt ) sin c ( xt ) d t + \frac{x}{\sqrt{π}} {\sum }_{n = 0}^{N} {\int }_{0}^{∞} {a}_{n} cos ( \frac{n π}{{τ}_{m}} t ) exp ( - yt ) sin c ( xt ) d t .$$

Each integral term can be found analytically. This leads to the approximation (15). It should be noted that at y>1 or x>4.5, the approximation (15) provides highly accurate results.

Using the Maclaurin expansion series near t=0, we can expand the arctangent function as follows

$$arctan ( \frac{t - x}{y} ) \approx arctan ( - \frac{x}{y} ) + \frac{y}{{x}^{2} + {y}^{2}} t + \frac{xy}{{( {x}^{2} + {y}^{2} )}^{2}} {t}^{2} . \qquad Eqn . ( 38 )$$

Consequently, applying integration by parts in equation (4) we can write

$$\begin{matrix}I ( x , y ) = \frac{y}{π} \int {\int }_{- ∞}^{∞} \frac{exp ( - {t}^{2} )}{{y}^{2} + {( x - t )}^{2}} d t d x \\ = - \frac{1}{π} {\int }_{- ∞}^{∞} exp ( - {t}^{2} ) arctan ( \frac{t - x}{y} ) d t .\end{matrix} \qquad Eqn . ( 39 )$$

Since exp (−t²) is a rapidly damping function, we can make restriction over integration within a domain −t_{M}t≦t≦t_{M}, where t_{M} is a margin value that can be taken equal to 6 according to Milone et al. [14]. Such restriction is assumed to be as the right side of expansion (38) approximates reasonably well over this domain when a small number of the polynomial terms is used.

Thus, we can rewrite equation (39) as

$$I ( x , y ) \approx - \frac{1}{π} {\int }_{- {t}_{M}}^{{t}_{M}} exp ( - {t}^{2} ) arctan ( \frac{t - x}{y} ) d t . \qquad Eqn . ( 40 )$$

Substituting now (38) into (40) this expression results in

$$I ( x , y ) \approx \frac{arctan ( x \text{/} y ) erf ( {t}_{M} )}{\sqrt{π}} - \frac{xy [ - {t}_{M} {e}^{- {t}_{M}^{2}} + \sqrt{π} erf ( {t}_{M} ) \text{/} 2 ]}{{π ( {x}^{2} + {y}^{2} )}^{2}} .$$

Taking into account that at t_{M}=6, erf (t_{M}=6)≈1 and −t_{M} exp (−tₘ²)≈−1.3917×10⁻¹⁵<<π/2, we can simplify this approximation as given by equation (17). It should be noted, however, that equation (17) can also be obtained by replacing the lower −t_{M} and upper t_{M} limits of the integral (40) by −∞ and ∞.

These replacements can be justified in a similar way described above since the exponential function exp (−t²) very rapidly decreases with increasing t leading to

$${\int }_{- {t}_{M}}^{{t}_{M}} exp ( - {t}^{2} ) arctan ( \frac{t - x}{y} ) d t \approx {\int }_{- ∞}^{∞} exp ( - {t}^{2} ) arctan ( \frac{t - x}{y} ) d t .$$

The methodology described here can be applied in other applications where interpolation through the grid points is required. As an example, consider the plasma dispersion function Z (z) that is commonly used in plasma Physics involving small wave propagating though Maxwellian media [33]

Z(z)=i\sqrt{π}w(z),

where w (z) is the complex error function, defined as

$$w ( z ) = {e}^{- {z}^{2}} ( 1 + \frac{2 i}{z} {\int }_{0}^{z} {e}^{{t}^{2}} d t ) .$$

There is a relation between the Voigt and complex error functions. Specifically, the Voigt function represents the real part of the complex error function K(x, y)=Re [w(x, y)]. Furthermore, the complex probability function W(z) considered above is reduced to complex error function w (z) when parameter y=Im[z] is positive:

W(z)=w(z), Im[z]>0.

Generalizing the methodology above, we can define the mean value of the plasma dispersion function as follows

$$\begin{matrix}\overline{Z} ( x , y ) = \frac{1}{Δ x} {\int }_{{x}_{l}}^{{x}_{l + 1}} w ( x , y ) d x \\ = \frac{1}{Δ x} {\int }_{{x}_{l}}^{{x}_{l + 1}} K ( x , y ) d x + i \frac{1}{Δ x} {\int }_{{x}_{l}}^{{x}_{l + 1}} L ( x , y ) d x ,\end{matrix}$$

$$where$$

$$\begin{matrix}L ( x , y ) = Im [ w ( x , y ) ] \\ = \frac{1}{\sqrt{π}} {\int }_{0}^{∞} exp ( - {t}^{2} \text{/} 4 ) exp ( - yt ) sin ( xt ) d t .\end{matrix}$$

As the mean value of the plasma dispersion function Z̅(x, y) accounts for the area under the curve within the step Δx, it also provides essentially higher accuracy in computation. As we can see from this example, our approach can be potentially useful as a versatile approach in broader applications, not only in the models involving spectral line broadening effects.

### REFERENCES

[1] Hitschfeld W, Houghton J T. Radiative transfer in the lower stratosphere due to the 9.6 micron band of ozone. Quart J Roy Meteor Soc 1961; 87:569-77.
[2] Clough S A, Kneizys F X, Rothman L S, Gallery W O. Atmospheric spectral transmittance and radiance: FASCOD1B. Proc Soc Photo Opt Instrum Eng 1981; 277:152-66.
[3] Clough S A, Iacono M J, Moncet J-L. Line-by-line calculation of atmospheric fluxes and cooling rates: application to water vapor. J Geophys Res 1992; 97:15761-85.
[4] Shephard M W, Clough S A, Payne V H, Smith W L, Kireev S, Cady-Pereira K E. Performance of the line-by-line radiative transfer model (LBLRTM) for temperature and species retrievals: IASI case studies from JAIVEx. Atmos Chem Phys 2009; 9:7397-417.
[5] Scott N A, Chedin A. A fast line-by-line method for atmospheric absorption computations: the authomated atmospheric absorption atlas. J Appl Meteorol 1981; 20:802-12.
[6] Edwards D P. Atmospheric transmittance and radiance calculations using line-by-line computer models. SPIE Modelling of the Atmosphere 1988; 928:94-116.
[7] Edwards D P. GENLN2: A General Line-by-line Atmospheric Transmittance and Radiance Model. Version 3.0 Description and Users Guide 1992. http:nldr.library.ucar.edurepositorycollectionsTECH-NOTE-000-000-000-178
[8] Gordley L L, Marshall B T, Chu D A. Linepak: Algorithms for modeling spectral transmittance and radiance. J Quant Spectrosc Radiat Transfer 1994; 52:563-80.
[9] Quine B M, Drummond J R. GENSPECT: a line-by-line code with selectable interpolation error tolerance. J Quant Spectrosc Radiat Transfer 2002; 74:147-65.
[10] Kratz D P, Mlynczak M G, Mertens C J, Brindley H, Gordley L L, Martin-Torres J, Miskolczi F M, Turnere D D. An inter-comparison of far-infrared line-by-line radiative transfer models. J Quant Spectrosc Radiat Transfer 2005; 90:323-41.
[11] Clough S A, Shephard M W, Mlawer E J, Delamere J S, Iacono M J, Cady-Pereira K, Boukabara S, Brown P D. Atmospheric radiative transfer modeling: a summary of the AER codes 2005; 91:233-44.
[12] Humlí\check{c}ek J. An efficient method for evaluation of the complex probability function: The Voigt function and its derivatives. J Quant Spectrosc Radiat Transfer 1979; 21:309-13.
[13] Humlí\check{c}ek J. Optimized computation of the Voigt and complex probability functions. J Quant Spectrosc Radiat Transfer 1982; 27:437-44.
[14] Milone A A E, Milone L A, Bobato G E. Numerical evaluation of the line broadening function H(a,v). Astrophys Space Sci 1988; 147:229-34.
[15] Poppe G P M, Wijers C M J. More efficient computation of the complex error function. ACM Trans Math Softw 1990; 16:38-46.
[16] Poppe G P M, Wijers C M J. Algorithm 680: evaluation of the complex error function, ACM Trans Math Softw 1990; 161:47.
[17] Kuntz M. A new implementation of the Humlí\check{c}ek algorithm for the calculation of the Voigt profile function. J Quant Spectrosc Radiat Transfer 1997; 57:819-24.
[18] Wells R J. Rapid approximation to the Voigt/Faddeeva function and its derivatives. J Quant Spectrosc Radiat Transfer 1999; 62:29-48.
[19] Schreier F, Kohlert D. Optimized implementations of rational approximations—a case study on the Voigt and complex error function, Comp Phys Commun 2008; 179:457-65.
[20] Abrarov S M, Quine B M, Jagpal R K. Rapidly convergent series for high-accuracy calculation of the Voigt function. J Quant Spectrosc Radiat Transfer 2010; 111:372-5.
[21] Zaghloul M R, Ali A N. Algorithm 916: computing the Faddeyeva and Voigt functions. ACM Trans. Math. Software 2011; 38:15:1-22.
[22] Abrarov S M, Quine B M. Efficient algorithmic implementation of the Voigt/complex error function based on exponential series approximation. Appl Math Comp 2011; 218:1894-902.
[23] Abrarov S M, Quine B M. On the Fourier expansion method for highly accurate computation of the Voigt/complex error function in a rapid algorithm. arXiv: 1205.1768v1.
[24] Kamarujjama M, Khan N U, Kashmin T. New representations concerning the analysis of the Voigt functions. Amer J Math Math Sci 2012; 1:23-8.
[25] Pagnini G, Saxena R K. On Mellin-Barnes integral representation of Voigt profile function. Forum der Berliner Mathematische Gesellschaft 2012; 23:47-64.
[26] Rothman L S, Gordon I E, Barbe A et. al. The HITRAN 2008 molecular spectroscopic database, J Quant Spectrosc Radiat Transfer 2009; 110:533-72.
[27] Brüggemann D, Bollig M. An efficient algorithm for frequency integration of Voigt profiles, J Quant Spectrosc Radiat Transfer 1992; 48:111-4.
[28] Liou K N. An introduction to atmospheric radiation. 2^{nd} Ed., Academic Press, USA 2002.
[29] Armstrong B H, Nicholls B W. Emission, absorption and transfer of radiation in heated atmospheres. Pergamon Press, New York 1972.
[30] Kac M. Statistical independence in probability, analysis and number theory. Carus Monographs no. 12. Mathematical Association of America. Washington D.C. 1959, p. 5.
[31] Keshavamurthy R S, Geetha R S. New properties and approximations of Doppler broadening function using Steffensen's inequality. Nucl Sci Eng 2009; 162:192-9.
[32] Abrarov S M, Quine B M, Jagpal R K. On the equivalence of Fourier expansion and
Poisson summation formula for the series approximation of the exponential function. arXiv:1202.5457v1.
[33] Fried B D, Conte S D. The plasma dispersion function. New York, Academic Press 1961.

TABLE 1

| x | y | Approximation (6) | Approximation (10) | Relative error |
|---|---|---|---|---|
| 10 | 10 | 4.424082477803966E−1 | 4.424082477803966E−1 | 0 |
| 5 | 10 | 2.606889148242353E−1 | 2.606889148242352E−1 | 4.2588E−16 |
| 5 | 5 | 4.402939200044195E−1 | 4.402939200044194E−1 | 2.5215E−16 |
| 1 | 1 | 3.827507050266603E−1 | 3.827507048713985E−1 | 4.0565E−10 |
| 1 | 0.5 | 5.169359021818141E−1 | 5.169362207686490E−1 | 6.1630E−7 |
| 0.5 | 0.5 | 2.936158265309017E−1 | 2.936154438703086E−1 | 1.3033E−6 |
| 0.1 | 0.5 | 6.144406408078174E−2 | 6.144950034404170E−2 | 8.8467E−5 |
| 0.1 | 0.1 | 7.631596294913978E−2 | 8.937925721777650E−2 | 1.4616E−1 |
| 0.05 | 0.1 | 3.419767205755875E−2 | 4.478947438175537E−2 | 2.3648E−1 |
| 0.05 | 0.05 | 2.203330511609780E−2 | 4.726226653569697E−2 | 5.3381E−1 |

1. method for rapid and accurate quantitative approximation of spectroscopic absorption and emission processes in radiative transfer line-by-line calculations with enhanced accuracy and/or at reduced spectral resolution without loss of accuracy.

2. method for the approximation of the integrated Voigt (error function) and plasma dispersion functions at finite precision.

3. method for the approximation of the integrated functions at finite precision using mean values of the discrete integration interval.
